(19) United States
(12) Patent Application Publication
(10) Pub. No.: US 2026/0199394 A1
(43) Pub. Date: Jul. 16, 2026

(54) HUMAN CS1 CAR-T CELLS

(71) Applicant: GUANGZHOU LITAI BIOTECHNOLOGY CO., LTD., Guangzhou, Guangdong (CN)

(72) Inventors: Lijun WU, Richmond, CA (US); Vita GOLUBOVSKAYA, Richmond, CA (US)

(21) Appl. No.: 19/133,666

(22) PCT Filed: Nov. 29, 2023
(86) PCT No.: PCT/CN2023/135139

§ 371 Date: May 28, 2025

Related U.S. Application Data
(60) Provisional application No. 63/385,934, filed on Dec. 2, 2022.

(51) Int. Cl. A61K 35/17; A61K 40/11; A61K 40/15; A61K 40/31; A61K 40/42; C07K 14/705; C07K 16/28

(52) U.S. Cl. CPC A61K 35/17; A61K 40/11; A61K 40/15; A61K 40/31; A61K 40/4202; C07K 14/7051; C07K 14/70521; C07K 14/70578; C07K 16/2803; A61K 2239/13; A61K 2239/21; C07K 2317/56; C07K 2317/622

(57) ABSTRACT

Provided herein is an anti-human CS1 single-chain variable fragment (scFv), comprising (i) VH having the amino acid of SEQ ID NO: 4 and VL having the amino acid of SEQ ID NO: 5, or (ii) VH having the amino acid of SEQ ID NO: 9 and VL having the amino acid of SEQ ID NO: 10. Provided herein is a chimeric antigen receptor fusion protein comprising from N-terminus to C-terminus: (i) a single-chain variable fragment (scFv) of the present invention, (ii) a transmembrane domain, (iii) at least one co-stimulatory domains, and (iv) an activating domain. The two CS1 CAR-T cells of the present invention demonstrate activity.

### CROSS-REFERENCE TO RELATED APPLICATIONS

The present application is a national stage of International Application No. PCT/CN2023/135139, filed on Nov. 29, 2023, which claims priority to U.S. Provisional Application No. 63/385,934, filed on Dec. 2, 2022. The disclosures of International Application No. PCT/CN2023/135139 and U.S. Provisional Application No. 63/385,934 are hereby incorporated by reference in their entireties.

### REFERENCE TO AN ELECTRONIC SEQUENCE LISTING

The contents of the electronic sequence listing (SequenceListing.xml; Size: 29,223 bytes; and Date of Creation: May 28, 2025) is herein incorporated by reference.

### FIELD OF THE INVENTION

The present invention relates to human CS1 antibody and human CS1-ScFv-CAR-T cells, which are useful in the field of adoptive immunity gene therapy for tumors.

### BACKGROUND OF THE INVENTION

Immunotherapy is emerging as a highly promising approach for the treatment of cancer. T cells or T lymphocytes, the armed forces of our immune system, constantly look for foreign antigens and discriminate abnormal (cancer or infected cells) from normal cells. Genetically modifying T cells with CAR (chimeric antigen receptor) constructs is the most common approach to design tumor-specific T cells. CAR-T cells targeting tumor-associated antigens (TAA) can be infused into patients (called adoptive cell transfer or ACT) representing an efficient immunotherapy approach [1, 2]. The advantage of CAR-T technology compared with chemotherapy or antibody is that reprogrammed engineered T cells can proliferate and persist in the patient (“a living drug”) [3], [4], (1).

CARs usually consist of a monoclonal antibody-derived single-chain variable fragment (scFv) at the N-terminal part, hinge, transmembrane domain and a number of intracellular co-activation domains: (i) CD28, (ii) CD137 (4-1BB), CD27 or other co-stimulatory domains, in tandem with a activation CD3-zeta domain. (FIG. 1) (2, 3). The evolution of CARs went from first generation (with no costimulatory domains) to second generation (with one co-stimulatory domain) to third generation CAR (with several costimulatory domains). Generating CARs with multiple costimulatory domains (the so-called 3^{rd} generation CAR) have led to increased cytolytic CAR-T cell activity, improved persistence of CAR-T cells leading to its augmented antitumor activity. The fourth and fifth generation of CAR included additional cytokines or cytokine receptors (4).

CS1 (SLAM family member 7, CD319) antigen is overexpressed in multiple myeloma cells. FIG. 2 shows the amino acid sequence of CS1 protein, in which the extracellular domain is bolded and underlined.

### BRIEF DESCRIPTION OF THE DRAWINGS

FIG. 1 shows the structures of CAR. The left panel: the first generation (no co-stimulation domains). The middle panel: the second generation (one co-stimulatory domain CD28 or 4-BB). The right panel: the third generation (two or several co-stimulatory domains). The Figure is from Golubovskaya (4).

FIG. 2 shows the Amino acid sequence of CS1 protein (SEQ ID NO: 1).

FIG. 3 shows the structure of CS1 CAR construct. The second-generation CAR is used with 4-1BB co-stimulatory domain.

FIG. 4 shows detection of CAR-positive T cells by FACS with anti-human FAB antibody.

FIGS. 5A-5B show that CS1-CAR-T cells (PMC1591) killed CHO-CS1 cells and did not kill CHO cells. 5A shows RTCA cytotoxicity assay with CAR-T cells (PMC 1591) effector cells and CHO-CS1 target cells. FIG. 5B shows cytotoxicity RTCA assay with CAR-T cells (PMC1591) and CHO target cells.

FIGS. 6A-6B show that CS1-CAR-T cells (PMC1594) killed CHO-CS1 cells and did not kill CHO cells. FIG. 6A shows RTCA cytotoxicity assay with CAR-T cells (PMC1594) effector cells and CHO-CS1 target. FIG. 6B shows cytotoxicity RTCA assay with CAR-T cells (PMC1594) and CHO target cells.

FIGS. 7A-7B show that CS1-CAR-T cells secrete high level of IFN-gamma against CHO-CS1 cells (7A), but not against CHO cells (7B).

FIG. 8A shows images of RPMI8226 xenograft tumors in NSG mice after treatment with CS1-CAR-T cells (PMC1591 and PMC1594) and control PBS. FIG. 8B shows that total flux was decreased after RPMI8226 cell injection into NSG mice. X-axis: days after RPMI8226 cells injection into NSG mice. Y-axis: photons/sec.

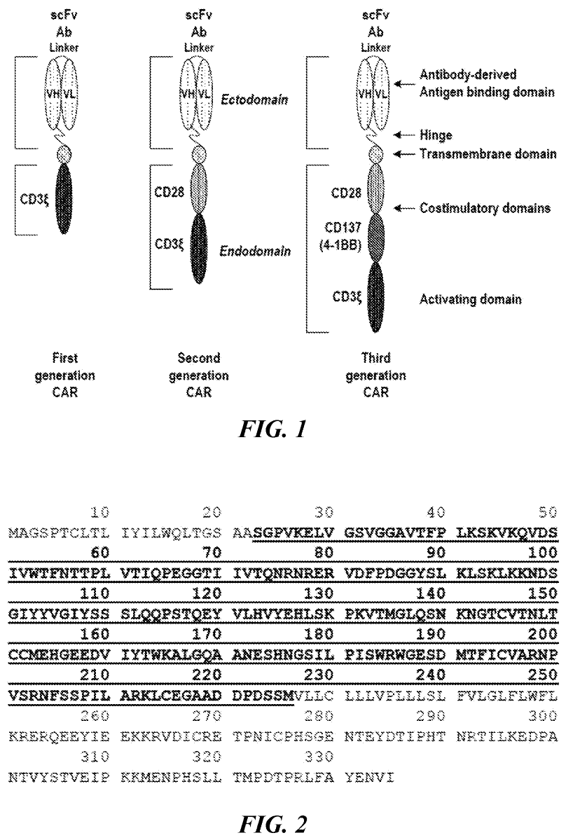

### DETAILED DESCRIPTION OF THE INVENTION

### Definitions

As used herein, a “chimeric antigen receptor (CAR)” is a receptor protein that has been engineered to give T cells the new ability to target a specific protein. The receptor is chimeric because they combine both antigen-binding and T-cell activating functions into a single receptor. CAR is a fused protein comprising an extracellular domain capable of binding to an antigen, a transmembrane domain, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell.

As used herein, a “domain” means one region in a polypeptide which is folded into a particular structure independently of other regions.

As used herein, a “single chain variable fragment (scFv)” means a single chain polypeptide derived from an antibody which retains the ability to bind to an antigen. An example of the scFv includes an antibody polypeptide which is formed by a recombinant DNA technique and in which Fv regions of immunoglobulin heavy chain (H chain) and light chain (L chain) fragments are linked via a spacer sequence. Various methods for engineering an scFv are known to a person skilled in the art.

As used herein, a “tumor antigen” means a biological molecule having antigenicity, expression of which causes cancer.

The inventors have screened human antibody library and provided human VH and VL and ScFV against CS1 antigen. The inventors have produced human CS1 ScFv-CAR-T cells to target cancer cells overexpressing CS1 tumor antigen. The CS1-CAR-T cells of the present invention have high cytotoxic activity against CS1-positive cell lines.

The present invention is directed to a human anti-human CS1 antibody, or an antigen-binding fragment thereof. The monoclonal anti-human CS1 antibody was generated against human CS1. In one embodiment, the monoclonal anti-human CS1 antibody, or an antigen-binding fragment thereof, is a single-chain variable fragment (scFv).

In one embodiment, the human anti-human CS1 antibody, or an antigen-binding fragment thereof, comprises VH having the amino acid of SEQ ID NO: 4 and V_{L} having the amino acid of SEQ ID NO: 5.

In another embodiment, the human anti-human CS1 antibody, or an antigen-binding fragment thereof, comprises VH having the amino acid of SEQ ID NO: 9 and V_{L} having the amino acid of SEQ ID NO: 10.

The present invention is also directed to a chimeric antigen receptor fusion protein comprising from N-terminus to C-terminus: (i) a single-chain variable fragment (scFv) against CS1, (ii) a transmembrane domain, (iii) at least one co-stimulatory domains, and (iv) an activating domain.

The inventors have produced CS1-CAR-T cells to target cancer cells overexpressing CS1 tumor antigen. The CS1-CAR-T cells of the present invention have high cytotoxic activity against hematological cancer cells.

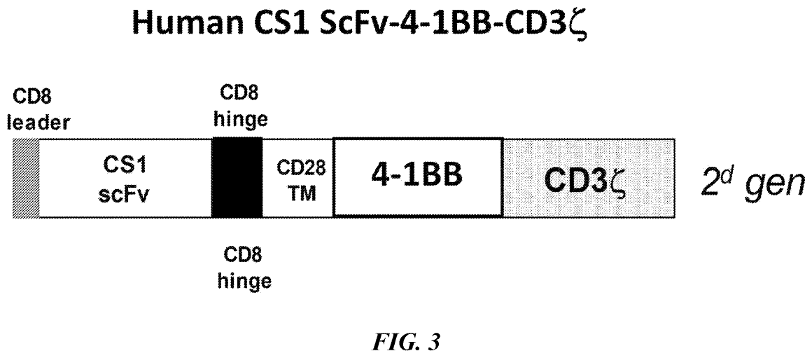

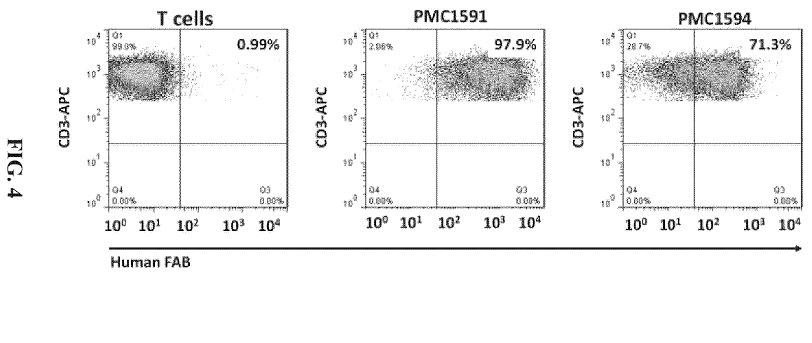

In some embodiments, the CAR structures are shown in FIG. 3.

In one embodiment, the co-stimulatory domain is selected from the group consisting of CD28, 4-1BB, GITR, ICOS-1, CD27, OX-40 and DAP10. A preferred the co-stimulatory domain is CD28 or 4-1BB.

A preferred activating domain is CD3 zeta (CD3 Z or CD3ζ).

The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor α or β chain, a CD3 zeta chain, CD28, CD3ε, CD45, CD4, CD5, CD8, CD9, CD16, CD22, CD33, CD37, CD64, CD80, CD86, CD134, CD137, ICOS, CD154, or a GITR can be used. The artificially designed transmembrane domain is a polypeptide mainly comprising hydrophobic residues such as leucine and valine. It is preferable that a triplet of phenylalanine, tryptophan and valine is found at each end of the synthetic transmembrane domain. Optionally, a short oligopeptide linker or a polypeptide linker, for example, a linker having a length of 2 to 10 amino acids can be arranged between the transmembrane domain and the intracellular domain. In one embodiment, a linker sequence having a glycine-serine continuous sequence can be used.

The present invention provides a nucleic acid encoding the CD37-CAR. The nucleic acid encoding the CAR can be prepared from an amino acid sequence of the specified CAR by a conventional method. A base sequence encoding an amino acid sequence can be obtained from the aforementioned NCBI RefSeq IDs or accession numbers of GenBank for an amino acid sequence of each domain, and the nucleic acid of the present invention can be prepared using a standard molecular biological and/or chemical procedure. For example, based on the base sequence, a nucleic acid can be synthesized, and the nucleic acid of the present invention can be prepared by combining DNA fragments which are obtained from a cDNA library using a polymerase chain reaction (PCR).

A nucleic acid encoding the CAR of the present invention can be inserted into a vector, and the vector can be introduced into a cell. For example, a virus vector such as a retrovirus vector (including an oncoretrovirus vector, a lentivirus vector, and a pseudo type vector), an adenovirus vector, an adeno-associated virus (AAV) vector, a simian virus vector, a vaccinia virus vector or a sendai virus vector, an Epstein-Barr virus (EBV) vector, and a HSV vector can be used. A virus vector lacking the replicating ability so as not to self-replicate in an infected cell is preferably used.

For example, when a retrovirus vector is used, a suitable packaging cell based on a LTR sequence and a packaging signal sequence possessed by the vector can be selected for preparing a retrovirus particle using the packaging cell. Examples of the packaging cell include PG13 (ATCC CRL-10686), PA317 (ATCC CRL-9078), GP+E-86 and GP+envAm-12, and Psi-Crip. A retrovirus particle can also be prepared using a 293 cell or a 293T cell having high transfection efficiency. Many kinds of retrovirus vectors produced based on retroviruses and packaging cells that can be used for packaging of the retrovirus vectors are widely commercially available from many companies.

A CAR-T cell binds to a specific antigen via the CAR, thereby a signal is transmitted into the cell, and as a result, the cell is activated. The activation of the cell expressing the CAR is varied depending on the kind of a host cell and an intracellular domain of the CAR, and can be confirmed based on, for example, release of a cytokine, improvement of a cell proliferation rate, change in a cell surface molecule, or the like as an index. For example, release of a cytotoxic cytokine (IFN-gamma, a tumor necrosis factor, lymphotoxin, etc.) from the activated cell causes destruction of a target cell expressing an antigen. In addition, release of a cytokine or change in a cell surface molecule stimulates other immune cells, for example, a B cell, a dendritic cell, a NK cell, and a macrophage.

The cell expressing the CAR can be used as a therapeutic agent for a disease. The therapeutic agent comprises the cell expressing the CAR as an active ingredient, and it may further comprise a suitable excipient.

The inventors have generated human CST-ScFv-4-1BB-CD3-CAR-T (CS1-CAR-T) cells against hematological cancer cells overexpressing CST (multiple myeloma). Human CS1-CAR-T cells express significantly higher cytotoxic activity against CST-positive cancer cells than against non-transduced T cells and mock-CAR-T cells.

The advantage of the human CST monoclonal antibody or CST-ScFv of the present invention over other known CS1 antibodies is that the present antibody has human antibody sequence and will not generate immune response.

The present CST antibody can be used for immunotherapy applications: toxin/drug-conjugated antibody, monoclonal therapeutic antibody, CAR-T cell immunotherapy.

CST-CAR-T cells using the present CST antibody can effectively target CST antigen in CS1-positive cell lines.

CS1-CAR-T can be used in combination with different chemotherapies such as checkpoint inhibitors, targeted therapies, small molecule inhibitors, and antibodies.

CS1-CAR-T cells can be used clinically against CS1-positive cells.

Modifications of co-activation domains (CD28, 28/4-1BB and others) can be used to increase the efficacy of CS1-CAR-T cells. Tag-conjugated CST scFv can be used for CAR generation.

Different co-activation signaling domains can be used with the present CS1-scFv inside CAR.

Fourth and fifth generation of CAR with addition different cytokines (IL-15, IL-12, IL-18, IL2β, or other) or cytokine receptors can be used for same CST scFvs inside CAR.

Combination of CS1-CAR with CARs targeting other tumor antigens or tumor microenvironment (VEGFR-1-3), PDL-1, CD80 or bispecific-scFv-CAR can be used to enhance activity of monotherapy CS1-CAR.

Bi-specific antibodies with CS1 and CD3 or other antigens can be generated for therapy.

The CS1-CAR can be used for generating other types of cells such as CS1-CAR-natural killer cells, gamma-delta T cells, iPS cells, CS1-CAR-macrophages, and other cells.

The following examples further illustrate the present invention. These examples are intended merely to be illustrative of the present invention and are not to be construed as being limiting.

### EXAMPLES

The inventors generated human CS1 CAR constructs inside lentiviral vector cloned into the lentiviral vector under MNDU3 promoter. The lentiviral CAR construct containing the CS1 ScFv-4-1BB-CD3zeta insert.

The lentiviruses were generated in 293 T cells and expression was validated by FACS.

### Example 1. CS1 ScFv Detected CS1 Antigen

We screened human antibody library and detected several human ScFv against CS1 antigen. We performed ELISA with human anti-CS1 and CS1 antigen. We detected several human CS1 ScFv binding CS1 antigen with ELISA OD at 450 nM>0.5 (=0.66).

### Example 2. Sequence of CS1 VH, VL, and ScFv

The sequences of CS1 VH and VL and ScFv were obtained by sequencing. We generated two CAR sequences with ScFv, PMC1591 (clone #4) and PMC1594 (clone #3).

### A. PMC1591

The following is the nucleotide sequence of PMC1591 human anti-CST scFv (SEQ ID NO: 2). The bold highlights the nucleotide sequence of PMC1591 V_{H}; the underlined highlights the nucleotide sequence of PMC1591 V_{L}; in between (shown in italics font) is the nucleotide sequence encoding a linker.

(SEQ ID NO: 2)
GAA GTG CAG CTG GTC CAG AGT GGC GCG GAG GTA AAG
AAG CCT GGA GCA TCA GTG AAA GTG TCC TGT AAA GCG
TCC GGA GGT ACA TTC TCT TCC TAT GCT ATC AGC TGG
GTA CGC CAG GCG CCT GGG CAA GGT CTG GAA TGG ATT
GGT TGG ATT TCC GCG AGT AAT GGA AAC ACC AAA TAT
GCC CAA AAA TTC CAA GGT AGA GTA ACC ATG ACT ACG
GAT ACC AGT ACT AGC ACC GCA TAT ATG GAG CTC CGG
AGC CTG ACC TCT GAC GAT ACT GCA GTA TTC TAT TGT
GCA CGC GCC GGA TCA ACA GGA GGA CAT TTC TAC TGG
GGC CAG GGA ACC TTG GTC ACG GTA AGC TCC GGC GGT
GGT GGG TCA GGA GGC GGC GGT TCC GGA GGT GGT GGA
AGT CAG AGT GTT GTG ACC CAA CCA CCA AGC GTC AGT
GCT GCT CCC GGG CAA AAA GTT ACA ATT TCC TGT AGC
GGG ACG TCC AGT AAC GTG GGG AAC AAC TAT GTG TCC
TGG TAC CAA AGA ATA CCT GGC ACT GCC CCA CGA TTG
CTT ATC TAT GAC GAC ACT AAT CGC CCG TCA GGT ATC
TCA GAT AGG TTC TCA GGC TCT AAG TCC GGT ACA AAG
GCC ACT TTG GCC ATC ACG GGT CTT CAA ACG GGT GAC
GAA GCC GAT TAT TTC TGC GGA ACT TGG CAT TCT GGT
CTG TCA GCG TAC GTG TTC GGC GCC GGC ACA AAG GTA
ACG GTT CTG

The following is the amino acid of PMC1591 human anti-human CST scFv (SEQ ID NO: 3).

(SEQ ID NO: 3)
EVQLVQSGAEVKKPGASVKVSCKASGGTFSSYAISWVRQAPGQGLEW
IGWISASNGNTKYAQKFQGRYTMTTDTSTSTAYMELRSLTSDDTAVF
YCARAGSTGGHFYWGQGTLVTVSSGGGGSGGGGSGGGGSQSVVTQPP
SVSAAPGQKVTISCSGTSSNVGNNYVSWYQRIPGTAPRLLIYDDTNR
PSGISDRFSGSKSGTKATLAITGLQTGDEADYFCGTWHSGLSAYVFG
AGTKVTVL

In SEQ ID NO: 3, the bold highlights the amino acid sequence of PMC1591 V_{H}.

(SEQ ID NO: 4)
EVQLVQSGAEVKKPGASVKVSCKASGGTFSSYAISWVRQAPGQGLEW
IGWISASNGNTKYAQKFQGRVTMTTDTSTSTAYMELRSLTSDDTAVF
YCARAGSTGGHFYWGQGTLVTVSS

In SEQ ID NO: 3, the underlined highlights the amino sequence of PMC1591 V_{L}.

(SEQ ID NO: 5)
QSVVTQPPSVSAAPGQKVTISCSGTSSNVGNNYVSWYQRIPGTAPRL
LIYDDTNRPSGISDRFSGSKSGTKATLAITGLQTGDEADYFCGTWHS
GLSAYVFGAGTKVTVL

In SEQ ID NO: 3, in between V_{H} and V_{L} (italicized) is the amino acid sequence of a linker sequence.

(SEQ ID NO: 6)
GGGGSGGGGSGGGGS

### B. PMC1594

The following is the nucleic acid sequence of PMC1594 human anti-CS1 scFv (SEQ ID NO: 7). The bold highlights the nucleotide sequence of PMC1594 VH; the underlined highlights the nucleotide sequence of PMC1594 VL; in between (shown in italics font) is the nucleotide sequence encoding a linker.

(SEQ ID NO: 7)
CAG GTA CAG CTC CAA CAA TGG GGA GCC GGG CTC GTT
AAA CCG AGC GAA ACA TTG AGC CTG ACA TGC GCC GTA
TAC GGG GGC AGT TTC AGT GGG TAC TAT TGG TCA TGG
ATA CGG CAG CCG CCC GGA AAG GGC CTC GAG TGG ATA
GGA GAG ATT AAC CAC TCC GGT AGT ACT AAC TAC AAC
CCA TCT CTG AAG AGC AGG GTA ACG ATC AGT GTT GAT
ACA TCC AAA AAT CAA TTT TCT CTT AAG CTC TCT TCC
GTC ACA GCA GCA GAT ACT GCT GTT TAC TAT TGC GCA
AGG GTG AGA AGA GGA GAT GCT TTC GAC ATA TGG GGC
CAA GGC ACC ATG GTT ACA GTG TCC AGT GGT GGT GGT
GGG TCC GGC GGG GGA GGC TCA GGT GGC GGC GGG TCA
GAA ATC GTT TTG ACG CAG AGT CCT TTG TCA CTG CCA
GTT ACT CCG GGT GAG CCG GCG TCC ATC AGC TGT AGG
TCT AGT CAA TCC CTG CTT CAT ATT AAC GGA TAC AAC
TAT TTG GAC TGG TAT CTC CAG AAA CCC GGA CAA AGT
CCC CAA CTC CTG ATC TAC TTG GGG TCA AAC AGG GCC
AGC GGG GTC CCA GAT CGC TTC TCC GGG TCT GGC AGT
GGG ACC GAC TTT ACG TTG AAG ATA AGC AGA GTG GAG
GCA GAA GAC GTA GGG GTC TAT TAC TGC ATG CAA GCT
CTG CAA ACA CAA GTG ACT TTC GGG CCA GGA ACT AAG
GTG GAT

The following is the amino acid sequence of PMC1594 human anti-human CS1 scFv (SEQ ID NO: 8).

(SEQ ID NO: 8)
QVQLQQWGAGLYKPSETLSLTCAVYGGSFSGYYWSWIRQPPGKGLEW
IGEINHSGSTNYNPSLKSRVTISVDTSKNQFSLKLSSVTAADTAVYY
CARVRRGDAFDIWGQGTMVTVSSGGGGSGGGGSGGGGSEIVLTQSPL
SLPVTPGEPASISCRSSQSLLHINGYNYLDWYLQKPGQSPQLLIYLG
SNRASGVPDRFSGSGSGTDFTLKISRVEAEDVGVYYCMQALQTQVTF
GPGTKVD

In SEQ ID NO: 8, the bold highlights the amino acid sequence of PMC1594 V_{H}.

(SEQ ID NO: 9)
QVQLQQWGAGLVKPSETLSLTCAVYGGSFSGYVWSWIRQPPGKGLEW
IGEINHSGSTNYNPSLKSRYTISVDTSKNQFSLKLSSVTAADTAVYY
CARVRRGDAFDIWGQGTMVTVSS

In SEQ ID NO: 8, the underlined highlights the amino acid sequence of PMC1594 V_{L}.

(SEQ ID NO: 10)
EIVLTQSPLSLPVTPGEPASISCRSSQSLLHINGYNYLDWYLQKPGQSP
QLLIYLGSNRASGVPDRFSGSGSGTDFTLKISRVEAEDVGVYYCMQALQ
TQVTFGPGTKVD

### Example 3. CS1-CAR Sequences

### A. PMC 1591

The scheme of CST-CAR (PMC1591) construct is shown on FIG. 3.

The following nucleotide sequence shows CST ScFv-CD8 hinge-CD28TM-4-1BB-CD3 zeta. The structure includes human CD8 signaling peptide, CST scFv (V_{H}-Linker-V_{L}), CD8 hinge, CD28 transmembrane, 4-1BB co-stimulatory domain and activation domain CD3 zeta.

The Nucleotide sequence of CS1 CAR (PMC1591) is shown below:

(SEQ ID NO: 11)
ATGGCCTTACCAGTGAC CGCCTTGCTCCTGCCGCTGGCCTTGCTGCTCCACGCCGCCAGGCCGGCTAGC
GAA GTG CAG CTG GTC CAG AGT GGC GCG GAG GTA AAG AAG CCT GGA GC
A TCA GTG AAA GTG TCC TGT AAA GCG TCC GGA GGT ACA TTC TCT TCC
TAT GCT ATC AGC TGG GTA CGC CAG GCG CCT GGG CAA GGT CTG GAA TG
G ATT GGT TGG ATT TCC GCG AGT AAT GGA AAC ACC AAA TAT GCC CAA
AAA TTC CAA GGT AGA GTA ACC ATG ACT ACG GAT ACC AGT ACT AGC AC
C GCA TAT ATG GAG CTC CGG AGC CTG ACC TCT GAC GAT ACT GCA GTA
TTC TAT TGT GCA CGC GCC GGA TCA ACA GGA GGA CAT TTC TAC TGG GG
C CAG GGA ACC TTG GTC ACG GTA AGC TCC GGC GGT GGT GGG TCA GGA
GGG GGG GGT TCC GGA GGT GGT GGA AGT CAG AGT GTT GTG ACC CAA CC
A CCA AGC GTG AGT GCT GCT CCC GGG CAA AAA GTT ACA ATT TGC TGT
AGC GGG ACG TCC AGT AAC GTG GGG AAC AAC TAT CTG TCC TGG TAC CA
A AGA ATA CCT GGC ACT GCC CCA CGA TTG CTT ATC TAT GAC GAC ACT
AAT CGC CCG TCA GGT ATC TCA GAT AGG TTC TCA GGC TCT AAG TCC GG
T ACA AAG GCC ACT TTG GCC ATC ACG GGT CTT CAA ACG GGT GAC GAA
GCC GAT TAT TTC TGC GGA ACT TGG CAT TCT GGT CTG TCA GCG TAC GT
G TTC GGG GCC GGG ACA AAG GTA ACG GTT CTG
CTCGAGAAGCC
CACCACGACGCCAGCGCCGCGACCACCAACACCGGCGCCCACCATCGCGTCGCAGCCCCT
GTCCCTGCGCCCAGAGGCGAGCCGGCCAGCGGCGGGGGGCGCAGTGCACACGAGGGGGCT
GGACTTCGCCAGTGATAAGCCCTTTTGGGTGCTGGTGGTGGTTGGTGGAGTCCTGGCTTG
CTATAGCTTGCTAGTAACAGTGGCCTTTATTATTTTCTGGGTGAAACGGGGCAGAAAGAA
ACTCCTGTATATATTCAAACAACCATTTATGAGACCAGTACAAACTACTCAAGAGGAAGA
TGGCTGTAGCTGCCGATTTCCAGAAGAAGAAGAAGGAGGATGTGAACTGAGAGTGAAGTT
CAGCAGGAGCGCAGACGCCCCCGCGTACCAGCAGGGCCAGAACCAGCTCTATAACGAGCT
CAATCTAGGACGAAGAGAGGAGTACGATGTTTTGGACAAGAGACGTGGCCGGGACCCTGA
GATGGGGGGAAAGCCGCAGAGAAGGAAGAACCCTCAGGAAGGCCTCTACAATGAACTGCA
GAAAGATAAGATGGCGGAGGCCTACAGTGAGATTGGGATGÅAAGGCGAGCGCCGGAGGGG
CAAGGGGCACGATGGCCTTTACCAGGGTCTCAGTACAGCCACCAAGGACACCTACGACGC
CCTTCACATGCAGGCCCTGCCCCCTCGCTAA

CD8 Signal Peptide-CS1 scFv (V_{H}-Linker-V_{L})-CD8 Hinge-CD28 TM-4-1BB-CD3-Zeta:

### <CD8 Leader)>

### Nucleotide

(SEQ ID NO: 12)
ATGGCCTTACCAGTGACCGCCTTGCTCCTGCCGCTGGCCTTGCTGCT
CCACGCCGCCAGGCCG

### Amino Acid

(SEQ ID NO: 13)
MALPVTALLLPLALLLHAARP

### <Nhe I Restriction Site for Cloning>

Nucleotide: GCTAGC
Amino Acid: AS

### <CS1 ScFv>

Nucleotide: SEQ ID NO: 2
Amino acid: see SEQ ID NO: 3

### <XhoI Restriction Site>

Nucleotide: CTCGAG
Amino Acid: LE

### <CD8 Hinge>

### Nucleotide

(SEQ ID NO: 14)
AAGCCCACCACGACGCCAGCGCCGCGACCACCAACACCGGCGCCCACCA
TCGCGTCGCAGCCCCTGTCCCTGCGCCCAGAGGCGAGCCGGCCAGCGGC
GGGGGGGGCAGTGCACACGAGGGGGCTGGACTTCGCCAGTGAT

### Amino Acid

(SEQ ID NO: 15)
KPTTTPAPRPPTPAPTIASQPLSLRPEASRPAAGGAVHTRGLDFASD

### <CD28 TM>

### Nucleotide

(SEQ ID NO: 16)
AAGCCCTTTTGGGTGCTGGTGGTGGTTGGTGGAGTCCTGGCTTGCTATAG
CTTGCTAGTAACAGTGGCCTTTATTATTTTCTGGGTG

(SEQ ID NO: 17)
KPFWVLVVVGGVLACYSLLVTVAFIIFWV

### <4-1BB Costimulatory Domain>

### Nucleotide

(SEQ ID NO: 18)
AACTACTCAAGAGGAAGATGGCTGTAGCTGCCGATTTCCAGAAGAAGAA
GAAGGAGGATGTGAACTG

### Amino Acid

(SEQ ID NO: 19)
KRGRKKLLYIFKQPFMRPVQTTQEEDGCSCRFPEEEEGGCEL

### <CD3 Zeta>

### Nucleotide

(SEQ ID NO: 20)
AGAGTGAAGTTCAGCAGGAGCGCAGACGCCCCCGCGTACCAGCAGGGC
CAGAACCAGCTCTATAACGAGCTCAATCTAGGACGAAGAGAGGAGTAC
GATGTTTTGGACAAGAGACGTGGCCGGGACCCTGAGATGGGGGGAAAG
CCGCAGAGAAGGAAGAACCCTCAGGAAGGCCTCTACAATGAACTGCAG
AAAGATAAGATGGCGGAGGCCTACAGTGAGATTGGGATGAAAGGCGAG
CGCCGGAGGGGCAAGGGGCACGATGGCCTTTACCAGGGTCTCAGTACA
GCCACCAAGGACACCTACGACGC
CCTTCACATGCAGGCCCTGCCCCCTCGCTAA

### Amino Acid

(SEQ ID NO: 21)
RVKFSRSADAPAYQQGQNQLYNELNLGRREEYDVLDKRRGRDPEMGGK
PQRRKNPQEGLYNELQKDKMAEAYSEIGMKGERRRGKGHDGLYQGLST
ATKDTYDALHMQALPPR

### Amino Acid Sequence of CS1-CAR Protein (PMC1591CAR)

(SEQ ID NO: 22)
MALPVTALLLPLALLLHAARPASEVQLVQSGAEVKKPGASVKVSCKASG
GTFSSYAISWVRQAPGQGLEWIGWISASNGNTKYAQKFQGRVTMTTDTS
TSTAYMELRSLTSDDTAVFYCARAGSTGGHFYWGQGTLVTVSSGGGGSG
GGGSGGGGSQSVVTQPPSVSAAPGQKVTISCSGTSSNVGNNYVSWYQRI
PGTAPRLLIYDDTNRPSGISDRFSGSKSGTKATLAITGLQTGDEADYFC
GTWHSGLSAYVFGAGTKVTVLLEKPTTTPAPRPPTPAPTIASQPLSLRP
EASRPAAGGAVHTRGLDFASDKPFWVLVVVGGVLACYSLLVTVAFIIFW
VKRGRKKLLYIFKQPFMRPVQTTQEEDGCSCRFPEEEEGGCELRVKFSR
SADAPAYQQGQNQLYNELNLGRREEYDVLDKRRGRDPEMGGKPQRRKNP
QEGLYNELQKDKMAEAYSEIGMKGERRRGKGHDGLYQGLSTATKDTYDA
LHMQALPPR

### B. PMC1594

The scheme of CS1-CAR (PMC1594) construct is shown on FIG. 3.

The following nucleotide sequence shows CST ScFv-CD8 hinge-CD28TM-4-1BB-CD3 zeta. The structure includes human CD8 signaling peptide, CST scFv (V_{H}-Linker-V_{L}), CD8 hinge, CD28 transmembrane, 4-1BB co-stimulatory domain and activation domain CD3 zeta.

### PMC1594 CAR Nucleotide:

(SEQ ID NO: 23)
atggccttaccagtgaccgccttgctcctgccgctggccttgctgctccacgccgccaggccggctagccaggtacagctccaacaat
ggggagccgggctcgttaaaccgagcgaaacattgagcctgacatgcgccgtatacgggggcagtttcagtgggtactattggtcat
ggatacggcagccgcccggaaagggcctcgagtggataggagagattaaccactccggtagtactaactacaacccatctctgaag
agcagggtaacgatcagtgttgatacatccaaaaatcaattttctcttaagctctcttccgtcacagcagcagatactgctgtttactatt
gcgcaagggtgagaagaggagatgctttcgacatatggggccaaggcaccatggttacagtgtccagtggtggtggtgggtccggcgg
gggaggctcaggtggcggcgggtcagaaatcgttttgacgcagagtcctttgtcactgccagttactccgggtgagccggcgtccatc
agctgtaggtctagtcaatccctgcttcatattaacggatacaactatttggactggtatctccagaaacccggacaaagtcccaactcc
tgatctacttggggtcaaacagggccagcggggtcccagatcgcttctccgggtctggcagtgggaccgactttacgttgaagataag
cagagtggaggcagaagacgtaggggtctattactgcatgcaagctctgcaaacacaagtgactttcgggccaggaactaaggtgga
tctcgagaagcccaccacgacgccagcgccgcgaccaccaacaccggcgcccaccatcgcgtcgcagcccctgtccctgcgccc
agaggcgagccggccagcggcgggggcgcagtgcacacgagggggctggacttcgccagtgataagcccttttgggtgctggt
ggtggttggtggagtcctggcttgctatagcttgctagtaacagtggcctttattattttctgggtgaaacggggcagaaagaaactcctg
tatatattcaaacaaccatttatgagaccagtacaaactactcaagaggaagatggctgtagctgccgatttccagaagaagaagaagg
aggatgtgaactgagagtgaagttcagcaggagcgcagacgcccccgcgtaccagcagggccagaaccagctctataacgagctc
aatctaggacgaagagaggagtacgatgttttggacaagagacgtggccgggaccctgagatggggggaaagccgcagagaagg
aagaaccctcaggaaggcctctacaatgaactgcagaaagataagatggcggaggcctacagtgagattgggatgaaaggcgagc
gccggaggggcaaggggcacgatggcctttaccagggtctcagtacagccaccaaggacacctacgacgcccttcacatgcaggc
cctgccccctcgctaa

PMC 1594 CAR Amino acid:

(SEQ ID NO: 24)
MALPVTALLLPLALLLHAARPASQVQLQQWGAGLVKPSETLSETCAVYGGSFSG
VYWSWIRQPPGKGLEWIGEINHSGSTNYNPSLKSRVTISVDTSKNQFSLKLSSVT
AADTAVYYCARVRRGDAFDIWGQGTMVTVSSGGGGSGGGGSGGGGSEIVLTQSP
LSLPVTPGEPASISCRSSQSLLHINGYNYLDWYLQKPGQSPQLLIYLGSNRASGVPDRF
SGSGSGTDFTLKISRVEAEDVGVYYCMQALQTQVTFGPGTKVDLEKPTTTPAPRPPT
PAPTIASQPLSLRPEASRPAAGGAVHTRGLDFASDKPFWVLVVVGGVLACYSLLVT
VAFIIFWVKRGRKKLLYIFKQPFMRPVQTTQEEDGCSCRFPEEEEGGCELRVKFSRSA
DAPAYQQGQNQLYNELNLGRREEYDVLDKRRGRDPEMGGKPQRRKNPQEGLY
NELQKDKMAEAYSEIGMKGERRRGKGHDGLYQGLSTATKDTYDALHMQALPP
R

### Example 4. Peripheral Blood Mononuclear Cell (PBMC) Isolation from Whole Blood

Whole blood (Stanford Hospital Blood Center, Stanford, CA) was collected from individual or mixed donors (depending on the amount of blood required) in 10 mL Heparin vacutainers (Becton Dickinson). Approximately 10 ml of whole anti-coagulated blood was mixed with sterile phosphate buffered saline (PBS) buffer for a total volume of 20 ml in a 50 ml conical centrifuge tube (PBS, pH 7.4, is without Ca⁺²/Mg⁺²). The layer of cells containing peripheral blood mononuclear cells (PBMC), seen at the diluted plasma/Ficoll interface was removed very carefully, avoiding any Ficoll, washed twice with PBS, and centrifuged at 200×g for 10 min at room temperature. Cells were counted with a hemocytomter. The PBMC were washed once with CAR-T media (AIM V-AlbuMAX (BSA) (Life Technologies), with 5% AB serum and 1.25 μg/mL amphotericin B (Gemini Bioproducts, Woodland, CA), 100 U/mL penicillin, and 100 μg/mL streptomycin) and used for experiments or were frozen at −80° C.

### Example 5. T-Cell Activation from PBMC

The isolated cells (washed with 1×PBS (pH7.4), no Ca²⁺/Mg²⁺) were washed once in CAR-T media (AIM V-AlbuMAX (BSA) (Life Technologies), with 5% AB serum and 1.25 μg/mL amphotericin B (Gemini Bioproducts, Woodland, CA), 100 U/mL penicillin, and 100 μg/mL streptomycin), in the absence of human interleukin-2 (huIL-2) (Invitrogen), at a concentration of 5×10⁵ cells/mL. The cells were resuspended to a final concentration of 5×10⁵ cells/mL in CAR-T medium with 300 U/mL human IL2. The PBMC was activated at a 1:1, CD3-CD28 bead-to-cell ratio.

### Example 6. T-Cell Transduction and Expansion

Following activation of PBMC, cells were incubated for 24 hr at 37° C., 5% CO₂. To each well of 1×10⁶ cells, 5×10⁶ lentivirus, and 2 μL/mL of media of Transplus (Alstem, Richmond, CA) (a final dilution of 1:500) were added. Cells were incubated for an additional 24 hours before repeating addition of virus. Cells were then grown in the continued presence of 300 U/mL of IL-2 Fresh medium with IL-2 for a period of 12-14 days (total incubation time was dependent on the final umber of CAR-T cells required). Cells concentrations were analyzed every 2-3 days, with media being added at that time to dilute the cell suspension to 1×10⁶ cells/mL

### Example 7. FACS for Detection of CAR-Positive Cells

Cells were washed and suspended in FACS buffer (Phosphate-buffered saline (PBS) plus 0.1% sodium azide and 0.4% BSA). Cells were divided them 1×10⁶ aliquots. Fc receptors were blocked with normal goat IgG (Life Technologies) in ice for 10 min. Biotin-labeled polyclonal goat anti-human-F(ab)2 antibodies (Life Technologies) were used to detect CS1 ScFv; biotin-labeled normal polyclonal goat IgG antibodies (Life Technologies) were used as an isotype control. (1:200 dilution, reaction volume of 100 μl). Cells were incubated at 4° C. for 25 minutes and washed once with FACS buffer.

Cells were suspended in FACS buffer and blocked with normal human IgG (Invitrogen) by adding 100 μl 1:1000 diluted normal human IgG to each tube. Cells were incubated in ice for 10 min, and washed with FACS buffer and re-suspended in 100 μl FACS buffer. Cells were then stained with phycoerythrin (PE)-labeled streptavidin (BD Pharmingen, San Diego, CA) and allophycocyanin (APC)-labeled CD3 (eBiocience, San Diego, CA). 1.0 μl PE and APC each was to tube 2 and 3.

### Example 8. Cytotoxicity Assay (Real-Time Cytotoxicity Assay)

The cytotoxicity was performed using ACEA machine according to manufacturer's protocol as described (5).

### Example 9. The CS1-CAR-T Cells Express CS1 ScFv

The CS1 scFv sequence was inserted with 4-1BB costimulatory and CD3 zeta activation domains inside CAR, and CAR lentiviruses were transduced into T cells. The CS1-CAR cells were effectively expanded in vitro (not shown). Mock control with scFv from intracellular protein were generated and used as a negative control in cytotoxicity and cytokine assay. The human CS1-Sc-Fv positive CAR-T cells were detected by FACS with anti-human FAB antibody (FIG. 4). The PMC1591 CAR-T cells were >95%-CAR positive and PMC1594 CAR-T cells were >70% CAR-positive after transduction with lentiviral CAR (FIG. 4).

### Example 10. CS1-CAR-T Cells Kill CHO-CS1 Cells

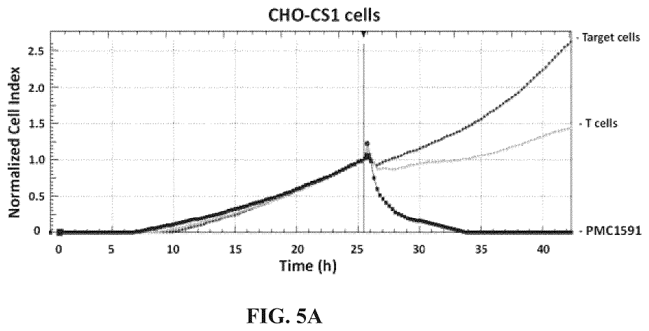

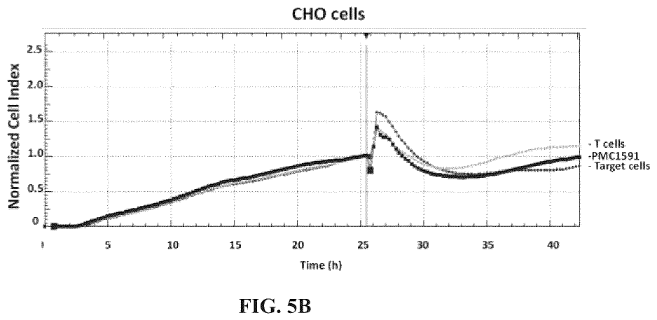

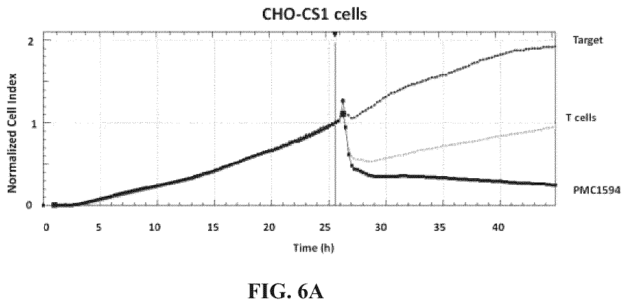

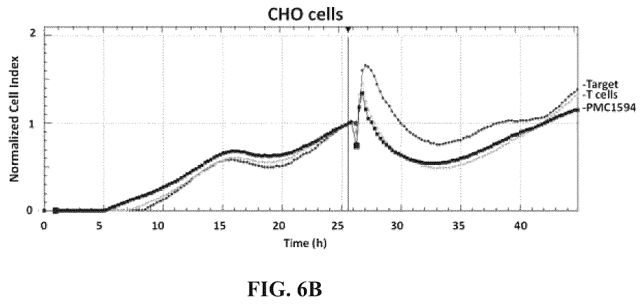

CS1-CAR-T cells (PMC1591) were co-incubated with CHO-CS1 stable cell lines and CHO cells (CS1-negative) and the Real-time cytotoxicity assay was performed using impedance-based XCelligence system. CS1-CAR-T cells killed CHO-CS1 cells better than T cells (FIG. 5A). CS1-CAR-T cells did not kill CHO (negative control cells (FIG. 5B). CS1-CAR-T cells (PMC1594) killed CHO-CS1 cells (FIG. 6A) and did not kill CHO cells (FIG. 6 B).

### Example 11. CS1-CAR-T Cells Secrete High Level of IFN-Gamma Against CS1-Positive Multiple Myeloma Cancer Cells

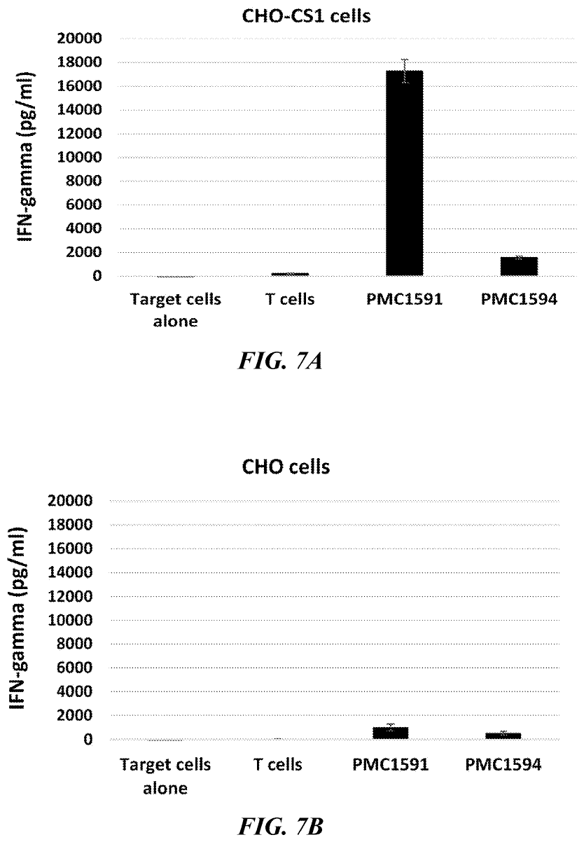

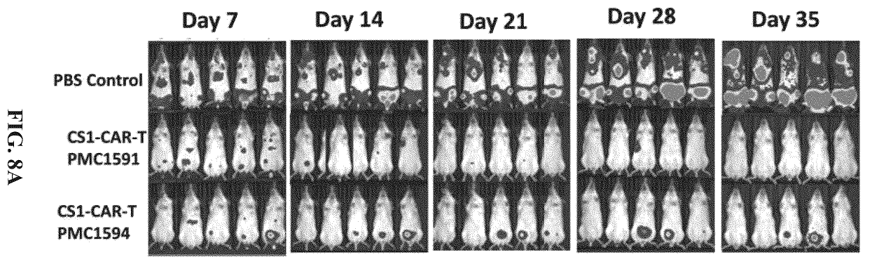

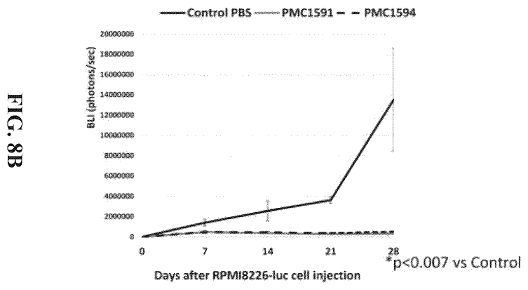

We collected supernatant of CS1-CAR-T incubated with CHO-CS1 cells and performed ELISA with kit from Fisher according to its protocol. The data show that CS1-4-1BB-CD3 CAR-T cells secreted IFN-gamma against CHO-CS1 cells (FIG. 7A) but not against CHO cells (FIG. 7B). The results show high specificity of CS1-CAR-T cells. PMC1591 CS1-CAR-T cells secreted more IFN-gamma than PMC1594 CS1-CAR-T cells.

### Example 12. CS1-4-1BB-CD3-CAR-T Cells Significantly Decrease RPMI8226 Multiple Myeloma Tumor Growth In Vivo

We used NSG mice and injected RPMI8226-luciferase positive cells intravenously into NSG mice. Next day we performed intravenous injections of 1×10⁷ CS1-CAR-T cells in NSG mice. The bioluminescnce signal was significantly decreased by CS1-41BB-CD3 cells corresponding to in vitro data (FIGS. 8A and 8B). The results demonstrate that CS1-41BB-CD3 CAR-T cells significantly decreased RPMI8226 multiple myeloma tumor growth. Similar results were shown with CS1-CAR-T cells (both PMC1591 and 1594) injected subcutaneously in that xenograft tumor growth was significantly decreased (data not shown).

### REFERENCES

1. S. A. Grupp, M. Kalos, D. Barrett, R. Aplenc, D. L. Porter, S. R. Rheingold, D. T. Teachey, A. Chew, B. Hauck, J. F. Wright, M. C. Milone, B. L. Levine and C. H. June: Chimeric antigen receptor-modified T cells for acute lymphoid leukemia. N Engl J Med, 368(16), 1509-18 (2013) doi:10.1056/NEJMoa1215134
2. M. V. Maus, A. R. Haas, G. L. Beatty, S. M. Albelda, B. L. Levine, X. Liu, Y. Zhao, M. Kalos and C. H. June: T cells expressing chimeric antigen receptors can cause anaphylaxis in humans. Cancer Immunol Res, 1, 26-31 (2013)
3. M. V. Maus, S. A. Grupp, D. L. Porter and C. H. June: Antibody-modified T cells: CARs take the front seat for hematologic malignancies. Blood, 123(17), 2625-35 (2014) doi:10.1182/blood-2013-11-492231
4. V. Golubovskaya: CAR-T Cells Targeting Immune Checkpoint Pathway Players. Front Biosci (Landmark Ed), 27(4), 121 (2022) doi:10.31083/j.fbl2704121

1. A human antibody against anti-human CS1 or an antigen-binding fragment thereof, comprising (i) VH having the amino acid of SEQ ID NO: 4 and VL having the amino acid of SEQ ID NO: 5; or (ii) VH having the amino acid of SEQ ID NO: 9 and VL having the amino acid of SEQ ID NO: 10.

2. A human single-chain variable fragment (scFv) against anti-human CS1, comprising VH having the amino acid of SEQ ID NO: 4 and VL having the amino acid of SEQ ID NO: 5.

3. The scFv of claim 2, further comprises a linker in between VH and VL.

4. The scFv of claim 3, which has the amino acid sequence of SEQ ID NO: 3.

5. (canceled)

6. A human single-chain variable fragment (scFv) against anti-human CS1, comprising VH having the amino acid of SEQ ID NO: 9 and VL having the amino acid of SEQ ID NO: 10.

7. The scFv of claim 6, further comprises a linker in between VH and VL.

8. The scFv of claim 7, which has the amino acid sequence of SEQ ID NO: 8.

9. A chimeric antigen receptor (CAR) comprising from N-terminus to C-terminus:
(i) the scFv of claim 2,
(ii) a transmembrane domain,
(iii) at least one co-stimulatory domains, and
(iv) an activating domain.

10. The CAR of claim 9 wherein the scFv has the amino acid sequence of SEQ ID NO: 3 or 8.

11. The CAR according to claim 9, wherein the co-stimulatory domain is CD28 or 4-1 BB.

12. The CAR according to claim 9, wherein the activation domain is CD3 zeta.

13. The CAR of claim 9, which has the amino acid sequence of SEQ ID NO: 22 or 24.

14. A nucleic acid encoding the CAR of claim 9.

15. T cells or natural killer cells modified to express the CAR of claim 9.

16. A chimeric antigen receptor (CAR) comprising from N-terminus to C-terminus:
(i) the scFv of claim 7,
(ii) a transmembrane domain,
(iii) at least one co-stimulatory domains, and
(iv) an activating domain.


SEQUENCE LISTING (24 sequences)

SEQ ID NO: 1 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 2 - DNA, 729 bp, Homo sapiens
  source 1..729: mol_type = other DNA
gaagtgcagctggtccagagtggcgcggaggtaaagaagcctggagcatcagtgaaagtgtcctgtaaagcgtccggaggtacattctcttcctatgctatcagctgggtacgccaggcgcctgggcaaggtctggaatggattggttggatttccgcgagtaatggaaacaccaaatatgcccaaaaattccaaggtagagtaaccatgactacggataccagtactagcaccgcatatatggagctccggagcctgacctctgacgatactgcagtattctattgtgcacgcgccggatcaacaggaggacatttctactggggccagggaaccttggtcacggtaagctccggcggtggtgggtcaggaggcggcggttccggaggtggtggaagtcagagtgttgtgacccaaccaccaagcgtcagtgctgctcccgggcaaaaagttacaatttcctgtagcgggacgtccagtaacgtggggaacaactatgtgtcctggtaccaaagaatacctggcactgccccacgattgcttatctatgacgacactaatcgcccgtcaggtatctcagataggttctcaggctctaagtccggtacaaaggccactttggccatcacgggtcttcaaacgggtgacgaagccgattatttctgcggaacttggcattctggtctgtcagcgtacgtgttcggcgccggcacaaaggtaacggttctg

SEQ ID NO: 3 - protein, 243 aa, Homo sapiens
EVQLVQSGAEVKKPGASVKVSCKASGGTFSSYAISWVRQAPGQGLEWIGWISASNGNTKYAQKFQGRVTMTTDTSTSTAYMELRSLTSDDTAVFYCARAGSTGGHFYWGQGTLVTVSSGGGGSGGGGSGGGGSQSVVTQPPSVSAAPGQKVTISCSGTSSNVGNNYVSWYQRIPGTAPRLLIYDDTNRPSGISDRFSGSKSGTKATLAITGLQTGDEADYFCGTWHSGLSAYVFGAGTKVTVL

SEQ ID NO: 4 - protein, 118 aa, Homo sapiens
EVQLVQSGAEVKKPGASVKVSCKASGGTFSSYAISWVRQAPGQGLEWIGWISASNGNTKYAQKFQGRVTMTTDTSTSTAYMELRSLTSDDTAVFYCARAGSTGGHFYWGQGTLVTVSS

SEQ ID NO: 5 - protein, 110 aa, Homo sapiens
QSVVTQPPSVSAAPGQKVTISCSGTSSNVGNNYVSWYQRIPGTAPRLLIYDDTNRPSGISDRFSGSKSGTKATLAITGLQTGDEADYFCGTWHSGLSAYVFGAGTKVTVL

SEQ ID NO: 6 - protein, 15 aa, synthetic construct
GGGGSGGGGSGGGGS

SEQ ID NO: 7 - DNA, 726 bp, Homo sapiens
  source 1..726: mol_type = other DNA
caggtacagctccaacaatggggagccgggctcgttaaaccgagcgaaacattgagcctgacatgcgccgtatacgggggcagtttcagtgggtactattggtcatggatacggcagccgcccggaaagggcctcgagtggataggagagattaaccactccggtagtactaactacaacccatctctgaagagcagggtaacgatcagtgttgatacatccaaaaatcaattttctcttaagctctcttccgtcacagcagcagatactgctgtttactattgcgcaagggtgagaagaggagatgctttcgacatatggggccaaggcaccatggttacagtgtccagtggtggtggtgggtccggcgggggaggctcaggtggcggcgggtcagaaatcgttttgacgcagagtcctttgtcactgccagttactccgggtgagccggcgtccatcagctgtaggtctagtcaatccctgcttcatattaacggatacaactatttggactggtatctccagaaacccggacaaagtccccaactcctgatctacttggggtcaaacagggccagcggggtcccagatcgcttctccgggtctggcagtgggaccgactttacgttgaagataagcagagtggaggcagaagacgtaggggtctattactgcatgcaagctctgcaaacacaagtgactttcgggccaggaactaaggtggat

SEQ ID NO: 8 - protein, 242 aa, Homo sapiens
QVQLQQWGAGLVKPSETLSLTCAVYGGSFSGYYWSWIRQPPGKGLEWIGEINHSGSTNYNPSLKSRVTISVDTSKNQFSLKLSSVTAADTAVYYCARVRRGDAFDIWGQGTMVTVSSGGGGSGGGGSGGGGSEIVLTQSPLSLPVTPGEPASISCRSSQSLLHINGYNYLDWYLQKPGQSPQLLIYLGSNRASGVPDRFSGSGSGTDFTLKISRVEAEDVGVYYCMQALQTQVTFGPGTKVD

SEQ ID NO: 9 - protein, 117 aa, Homo sapiens
QVQLQQWGAGLVKPSETLSLTCAVYGGSFSGYYWSWIRQPPGKGLEWIGEINHSGSTNYNPSLKSRVTISVDTSKNQFSLKLSSVTAADTAVYYCARVRRGDAFDIWGQGTMVTVSS

SEQ ID NO: 10 - protein, 110 aa, Homo sapiens
EIVLTQSPLSLPVTPGEPASISCRSSQSLLHINGYNYLDWYLQKPGQSPQLLIYLGSNRASGVPDRFSGSGSGTDFTLKISRVEAEDVGVYYCMQALQTQVTFGPGTKVD

SEQ ID NO: 11 - DNA, 1500 bp, Homo sapiens
  source 1..1500: mol_type = other DNA
atggccttaccagtgaccgccttgctcctgccgctggccttgctgctccacgccgccaggccggctagcgaagtgcagctggtccagagtggcgcggaggtaaagaagcctggagcatcagtgaaagtgtcctgtaaagcgtccggaggtacattctcttcctatgctatcagctgggtacgccaggcgcctgggcaaggtctggaatggattggttggatttccgcgagtaatggaaacaccaaatatgcccaaaaattccaaggtagagtaaccatgactacggataccagtactagcaccgcatatatggagctccggagcctgacctctgacgatactgcagtattctattgtgcacgcgccggatcaacaggaggacatttctactggggccagggaaccttggtcacggtaagctccggcggtggtgggtcaggaggcggcggttccggaggtggtggaagtcagagtgttgtgacccaaccaccaagcgtcagtgctgctcccgggcaaaaagttacaatttcctgtagcgggacgtccagtaacgtggggaacaactatgtgtcctggtaccaaagaatacctggcactgccccacgattgcttatctatgacgacactaatcgcccgtcaggtatctcagataggttctcaggctctaagtccggtacaaaggccactttggccatcacgggtcttcaaacgggtgacgaagccgattatttctgcggaacttggcattctggtctgtcagcgtacgtgttcggcgccggcacaaaggtaacggttctgctcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 12 - DNA, 63 bp, Homo sapiens
  source 1..63: mol_type = other DNA
atggccttaccagtgaccgccttgctcctgccgctggccttgctgctccacgccgccaggccg

SEQ ID NO: 13 - protein, 21 aa, Homo sapiens
MALPVTALLLPLALLLHAARP

SEQ ID NO: 14 - DNA, 141 bp, Homo sapiens
  source 1..141: mol_type = other DNA
aagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgat

SEQ ID NO: 15 - protein, 47 aa, Homo sapiens
KPTTTPAPRPPTPAPTIASQPLSLRPEASRPAAGGAVHTRGLDFASD

SEQ ID NO: 16 - DNA, 87 bp, Homo sapiens
  source 1..87: mol_type = other DNA
aagcccttttgggtgctggtggtggttggtggagtcctggcttgctatagcttgctagtaacagtggcctttattattttctgggtg

SEQ ID NO: 17 - protein, 29 aa, Homo sapiens
KPFWVLVVVGGVLACYSLLVTVAFIIFWV

SEQ ID NO: 18 - DNA, 126 bp, Homo sapiens
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - protein, 42 aa, Homo sapiens
KRGRKKLLYIFKQPFMRPVQTTQEEDGCSCRFPEEEEGGCEL

SEQ ID NO: 20 - DNA, 342 bp, Homo sapiens
  source 1..342: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 21 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - protein, 499 aa, Homo sapiens
MALPVTALLLPLALLLHAARPASEVQLVQSGAEVKKPGASVKVSCKASGGTFSSYAISWVRQAPGQGLEWIGWISASNGNTKYAQKFQGRVTMTTDTSTSTAYMELRSLTSDDTAVFYCARAGSTGGHFYWGQGTLVTVSSGGGGSGGGGSGGGGSQSVVTQPPSVSAAPGQKVTISCSGTSSNVGNNYVSWYQRIPGTAPRLLIYDDTNRPSGISDRFSGSKSGTKATLAITGLQTGDEADYFCGTWHSGLSAYVFGAGTKVTVLLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - DNA, 1497 bp, Homo sapiens
  source 1..1497: mol_type = other DNA
atggccttaccagtgaccgccttgctcctgccgctggccttgctgctccacgccgccaggccggctagccaggtacagctccaacaatggggagccgggctcgttaaaccgagcgaaacattgagcctgacatgcgccgtatacgggggcagtttcagtgggtactattggtcatggatacggcagccgcccggaaagggcctcgagtggataggagagattaaccactccggtagtactaactacaacccatctctgaagagcagggtaacgatcagtgttgatacatccaaaaatcaattttctcttaagctctcttccgtcacagcagcagatactgctgtttactattgcgcaagggtgagaagaggagatgctttcgacatatggggccaaggcaccatggttacagtgtccagtggtggtggtgggtccggcgggggaggctcaggtggcggcgggtcagaaatcgttttgacgcagagtcctttgtcactgccagttactccgggtgagccggcgtccatcagctgtaggtctagtcaatccctgcttcatattaacggatacaactatttggactggtatctccagaaacccggacaaagtccccaactcctgatctacttggggtcaaacagggccagcggggtcccagatcgcttctccgggtctggcagtgggaccgactttacgttgaagataagcagagtggaggcagaagacgtaggggtctattactgcatgcaagctctgcaaacacaagtgactttcgggccaggaactaaggtggatctcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctctacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 24 - protein, 498 aa, Homo sapiens
MALPVTALLLPLALLLHAARPASQVQLQQWGAGLVKPSETLSLTCAVYGGSFSGYYWSWIRQPPGKGLEWIGEINHSGSTNYNPSLKSRVTISVDTSKNQFSLKLSSVTAADTAVYYCARVRRGDAFDIWGQGTMVTVSSGGGGSGGGGSGGGGSEIVLTQSPLSLPVTPGEPASISCRSSQSLLHINGYNYLDWYLQKPGQSPQLLIYLGSNRASGVPDRFSGSGSGTDFTLKISRVEAEDVGVYYCMQALQTQVTFGPGTKVDLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR